(19) United States
(12) Patent Application Publication
(10) Pub. No.: US 2018/0228711 A1
(43) Pub. Date: Aug. 16, 2018

(54) 3-(4-HYDROXYPHENYL) PROPANOIC ACID AMIDE FOR USE IN TISSUE REPAIR AND/OR SKIN BRIGHTENING

(71) Applicant: NMETICS IVS, Risskov (DK)

(72) Inventor: Jan-Elo Bjarne Jørgensen, Skanderborg (DK)

(73) Assignee: NMETICS IVS, Risskov (DK)

(21) Appl. No.: 15/752,001

(22) PCT Filed: Aug. 12, 2016
(86) PCT No.: PCT/DK2016/050272

§ 371 Date: Feb. 12, 2018

(30) Foreign Application Priority Data
Aug. 12, 2015 (DK) PA 2015 70522

(51) Int. Cl. A61K 8/42; A61Q 19/02; A61K 31/165; A61P 17/02

(52) U.S. Cl. CPC A61K 8/42; A61P 17/02; A61K 31/165; A61Q 19/02

(57) ABSTRACT

In one aspect the present invention relates to 3-(4-hydroxyphenyl)propanoic acid amide (PA) for use as a tissue repair agent. In a second aspect, the present invention relates to the use of PA as a skin brightening/lightening agent.

### TECHNICAL FIELD OF THE INVENTION

The present invention relates to effects on human cells of the aromatic natural compound 3-(4-hydroxyphenyl)propanoic acid amide (PA) from apples (Malus domestica). In particular, the present invention relates to enhancement of cell motility and depigmentation effects potentially conferring improved cell tissue damage repair and lightening/brightening of skin colour.

### BACKGROUND OF THE INVENTION

The repair of cell tissue is a complex process involving several cell biochemical sub-processes. These involve disinfecting and cleaning up the damaged area by infusion of cytokines, migration of cells to cover up the damage as well as contraction and granulation of the new tissue. The cell migration primarily being driven by coordinated assembly and disassembly of actin filaments. Thus, improvement of the cells ability to adhere at focal points to the substratum and migrate more efficiently to recover the tissue damage will overall improve efficacy of the cell tissue repair process.

Freckles, chloasma and pigmentary deposits after sunburn tend to occur or increase or become difficult to disappear with increasing age, thus being one of serious problems on skin care to persons of the middle or advanced age.

Now, there is a high demand of medicines and/or cosmetics which act to restore acquired deposit of a pigment i.e. melanin to the normal skin colour. A number of medicines have been developed and put into practice. For instance, peroxides are believed to bleach melanin and thus attempts have been made in the use of hydrogen peroxide, zinc peroxide, sodium peroxide, benzoyl peroxide and the like. However, these peroxides are unstable compounds and little effects of reducing the pigmentation were not observed in practical application conditions.

In recent years, cosmetics, which comprise vitamin C (L-ascorbic acid) having good reducing ability were proposed, however, it showed little effects in external applications. Moreover, vitamin C is rather unstable and have disadvantage to be comprised in cosmetics. On the other hand, in Europe and the United States of America, hydroquinone and derivatives thereof, have been used for treatment of moth patches or bleaching of coloured/dark skin. However, these compounds have safety problems (such as high stimulative, allergic troubles and the like) and may sometimes cause white spots, thus, use of these substances as medicine/cosmetics being rather disadvantageous. Use of a variety of melanin inhibitors has been also reported but almost all the substances showed little melanin inhibiting effects.

3-(4-hydroxyphenyl)propanoic acid amide (PA) was originally detected as a compound in apple tree sap. This compound was purified, identified and subsequently synthesized chemically.

WO2007046721 discloses a method of manufacturing PA, its application in the manufacture of anti-aging compositions.

Improved cell migration process and cell depigmentation process would be advantageous for many applications, and in particular, a more efficient and/or reliable skin repair and skin colour brightening would be advantageous.

### SUMMARY OF THE INVENTION

The in here presented experimental data show that PA confers enhanced mobility abilities as well as decreasing melanin production in human cells in culture.

PA was tested in in vitro cell migration model systems, such as in vitro bi and uni-directional cell migration and collagen lattice contraction and Boyden chamber chemotaxic motility assay. PA pre-treatments were performed on cells that belonged to various ages of the lifespan resulting in development of greater contractile forces, improving cell locomotion, and faster migration abilities indicating ability to aid tissue repair in vivo (example 2).

PA was tested for melanin production effects as well. The pigmentation studies involved melanin pigments as the key determinants of skin color that is produced by melanocytes located within the basal layer separating the dermis and epidermis in the skin of the human body. The enzyme tyrosinase is responsible for catalyzing various steps in the melanin pigment biosynthetic pathway responsible for skin cell pigmentation. The potential skin lightening/brightening abilities was measured by PA pretreated murine melanocytes (example 3).

PA clearly promoted physiological alterations that breaks down tyrosinase. This inhibition of tyrosinase promotes de-pigmentation of skin indicating that PA is a powerful skin colour brightening agent.

PA has the further advantage that it is known to be present in plant juice and may therefore be considered a natural product

Thus, PA affects motility as well as melanin production in human cells.

Thus, an object of the present invention is to provide a compound, which may improve cell migration/motility in vivo and in vitro.

Another object is to provide a compound, which may improve skin brightening/skin lighting/skin whitening.

Thus, one aspect of the invention relates to a compound of the formula (I) or a composition comprising a compound of the formula (I):

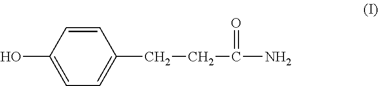 [SMILES: NC(=O)CCc1ccc(O)cc1]

for use as a tissue repair agent and/or, a tissue restitution agent e.g. muscle restitution of athletes.

Another aspect of the present invention relates to the use of a compound of the formula (I) or a composition comprising a compound of the formula (I):

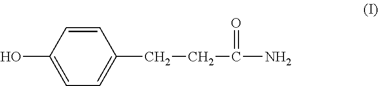 [SMILES: NC(=O)CCc1ccc(O)cc1]

as a skin brightening agent.

Yet another aspect of the present invention relates to the use of a compound of the formula (I) or a composition comprising a compound of the formula (I):

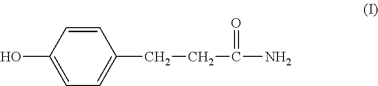 [SMILES: NC(=O)CCc1ccc(O)cc1]

as a tyrosinase inhibitor.

Still another aspect of the present invention is to provide a compound of the formula (I) or a composition comprising a compound of the formula (I):

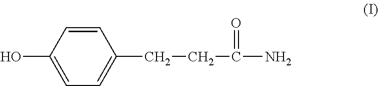 [SMILES: NC(=O)CCc1ccc(O)cc1]

for use as an agent to improve cell motility and/or surface cell migration

### BRIEF DESCRIPTION OF THE FIGS

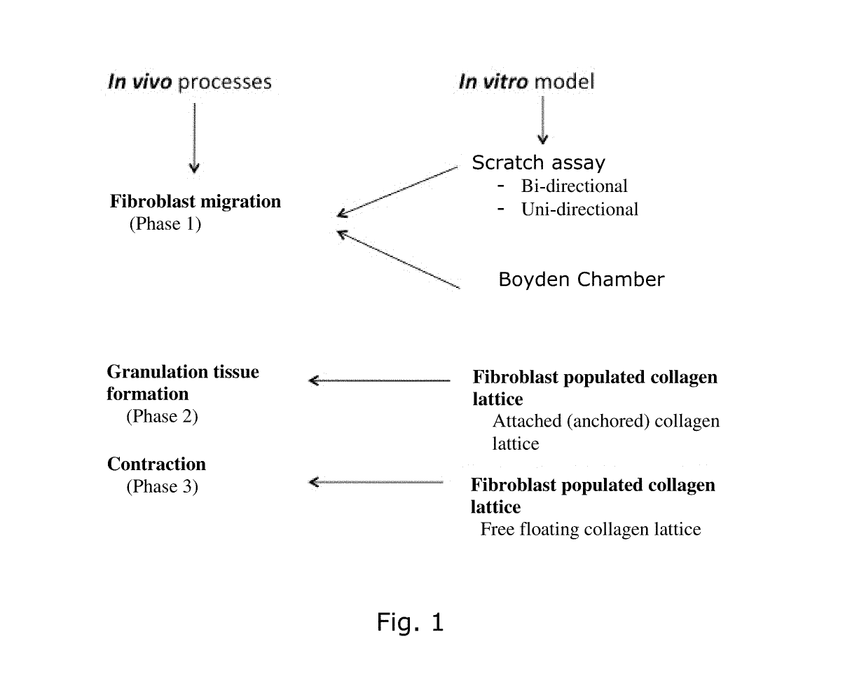

FIG. 1 shows in vitro assays reflecting tissue repair sub-processes in vivo.

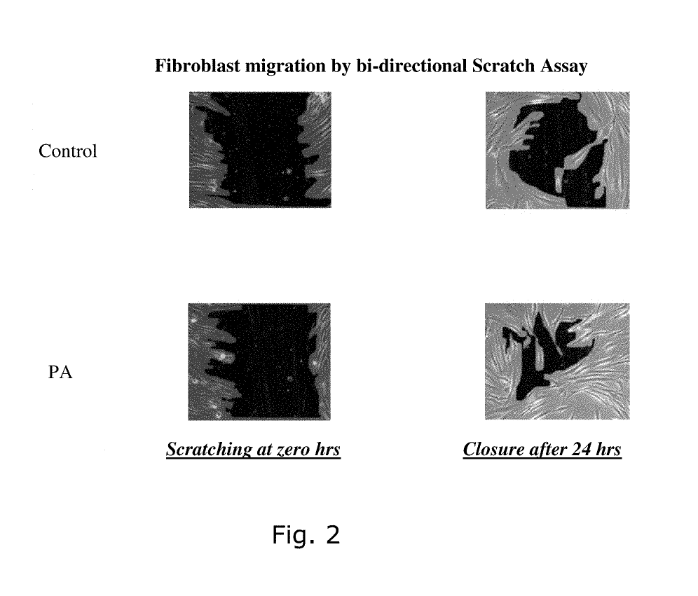

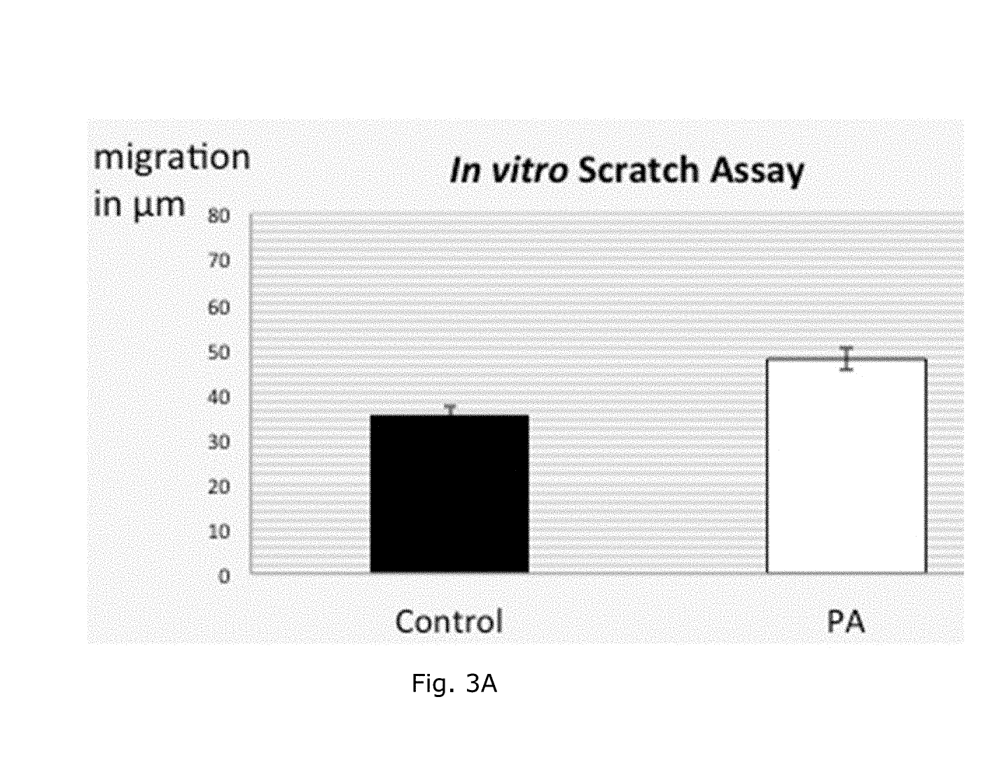

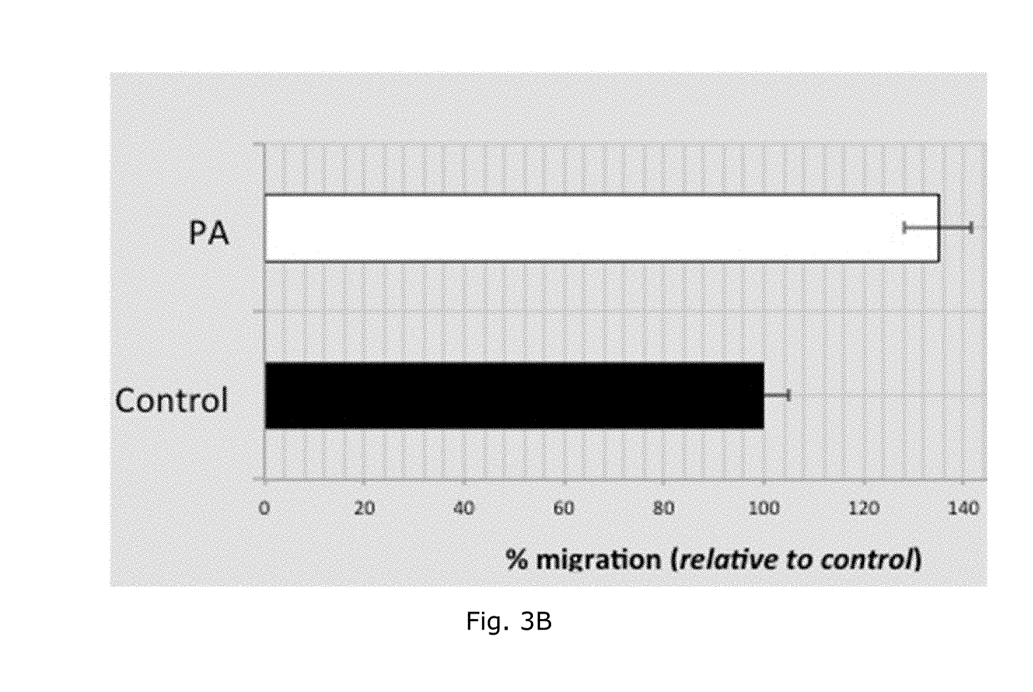

FIG. 2 shows the effect of PA treatment on the migration rate of human fibroblast cells in a bidirectional scratch assay. Fibroblasts were pre-treated with PA (80 μM) for three days before performing the scratch assay. Migration was quantified by a micrometer scale (see FIG. 3).

FIGS. 3A+3B show quantification of results of experiments from FIG. 2. PA improves tissue migration by about 37% over the untreated.

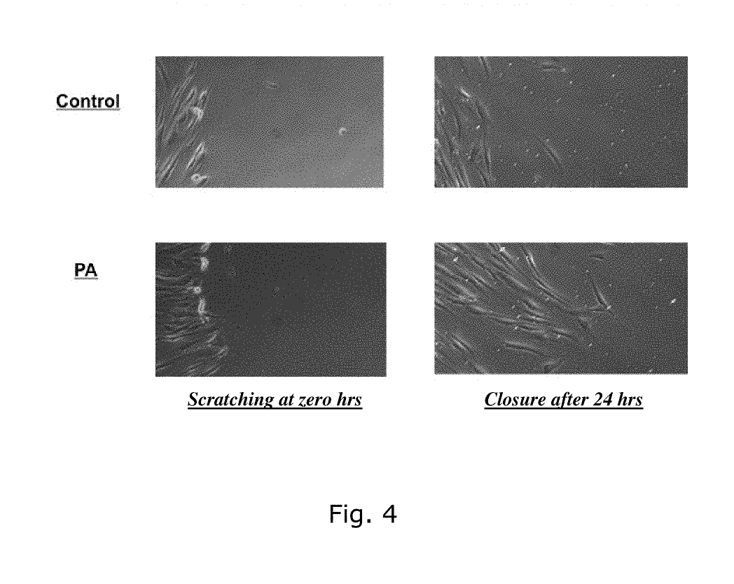

FIG. 4 shows the effect of PA treatment on the migration rate of human fibroblast cells in a uni-directional scratch assay. Fibroblasts were pre-treated with PA (80 μM) for three days before performing the scratch assay.

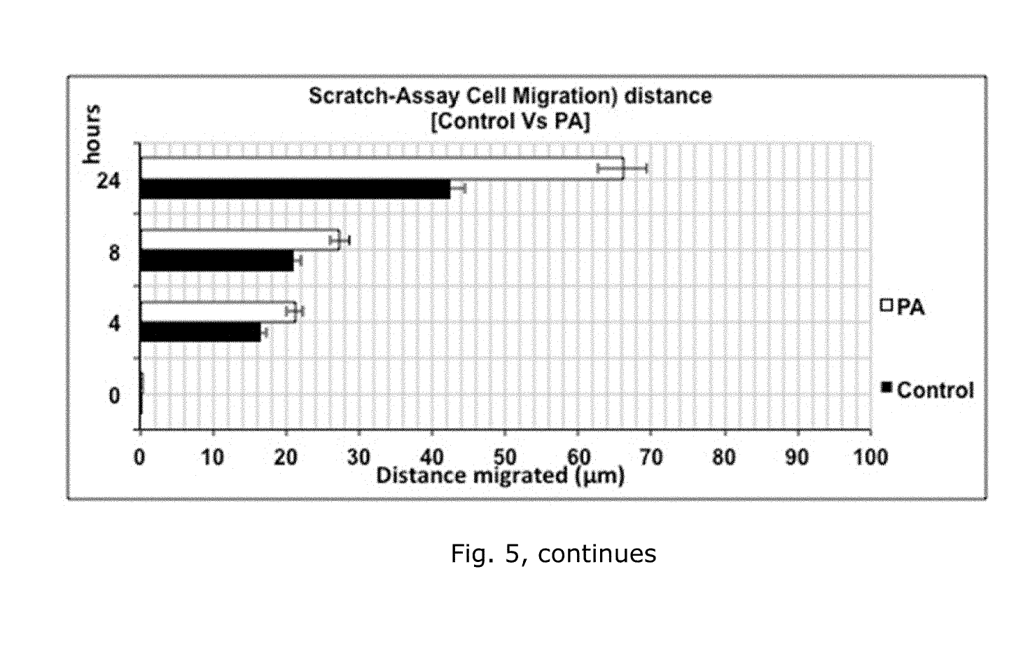

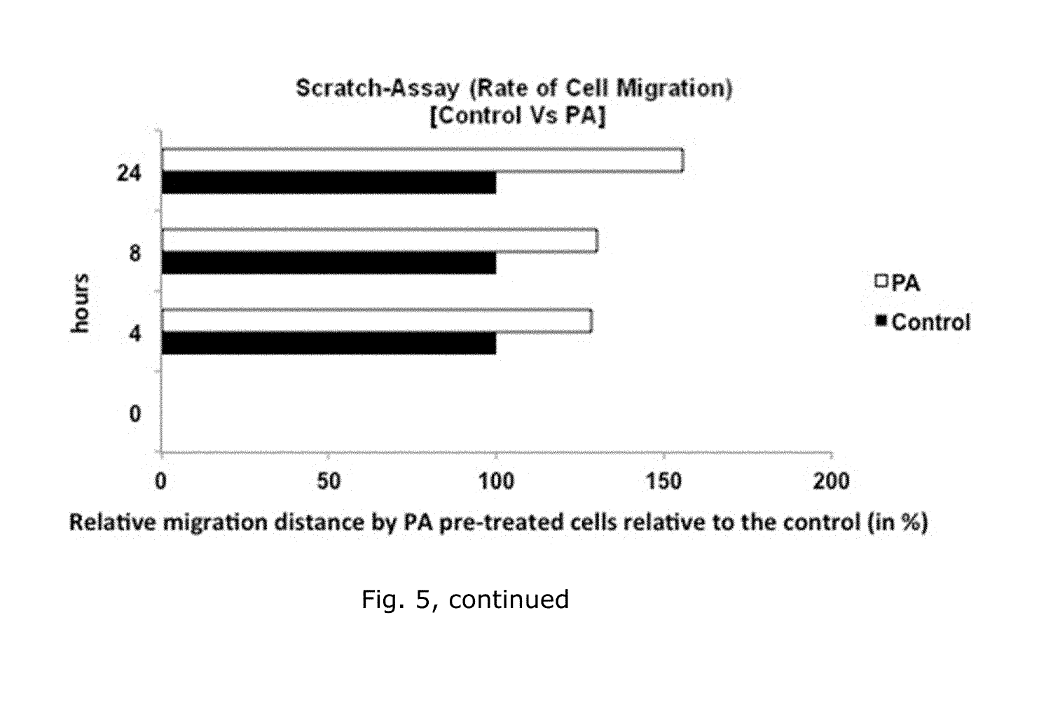

FIG. 5 shows quantification of results of experiments from FIG. 4. After 24 hrs PA enhances migration (tissue repair) in vitro by more than 60% as measured by uni-directional cell migration. Top: cell migration distance. Bottom: Rate of cell migration.

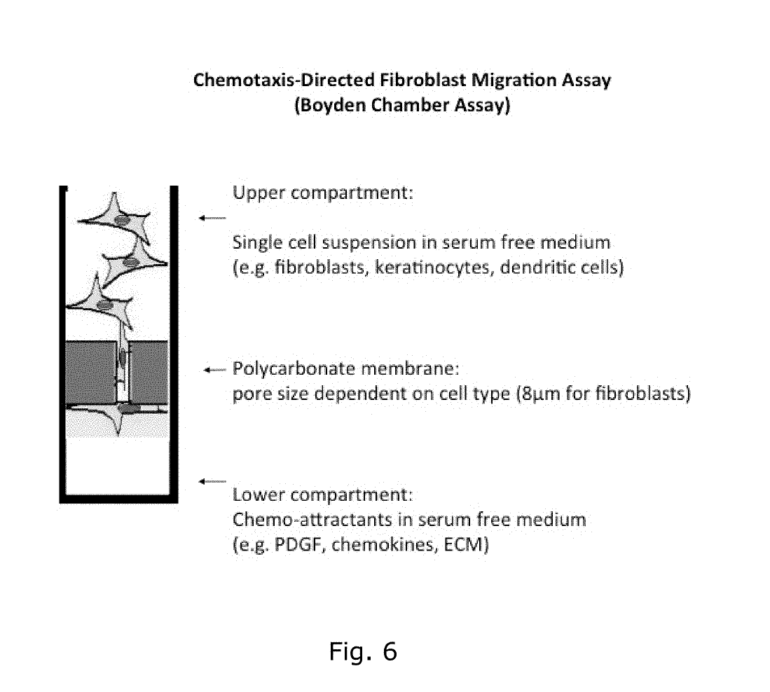

FIG. 6 illustrates the principle of the Boyden chamber assay for chemotaxic cell migration.

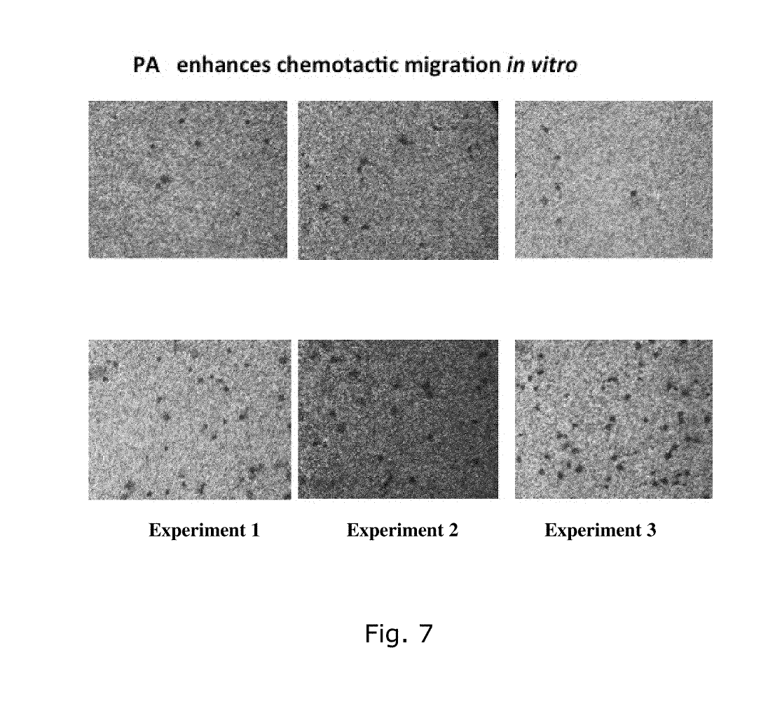

FIG. 7 shows the result of three independent Boyden chamber assays. Migrated cells become attached to a polycarbonate membrane at the bottom of the chamber and stained (dots). Three separate experiments are shown. Top: Controls. Bottom: PA treated.

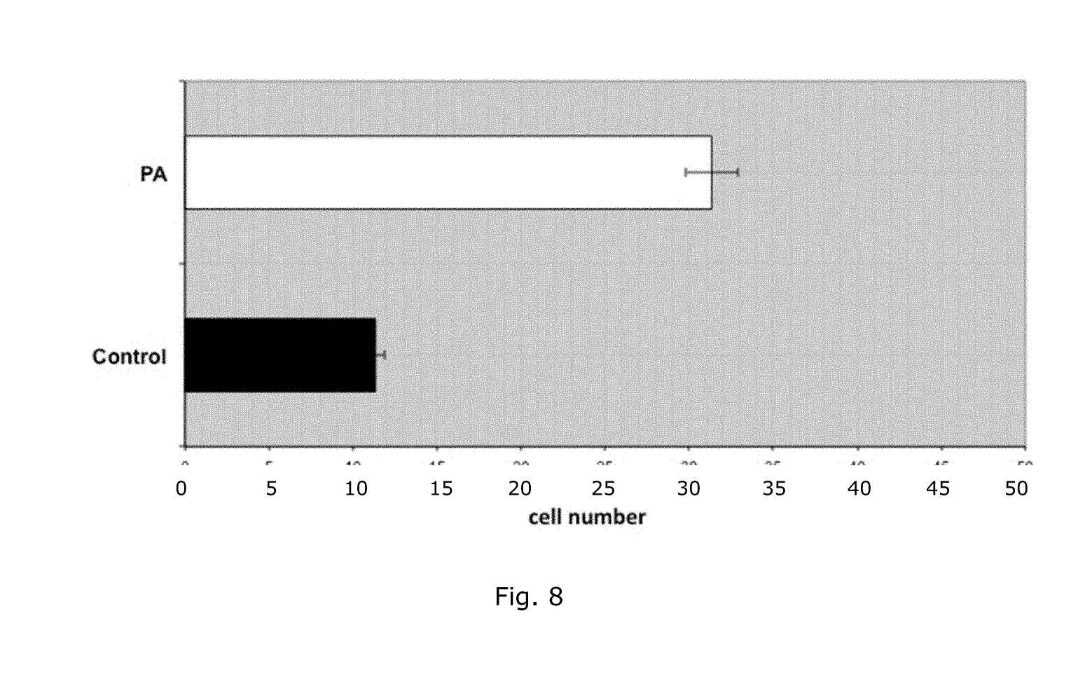

FIG. 8 is the quantification of the Boyden chamber assay results. PA enhances chemotactic migration towards chemo-attractants by a factor 2.7 in vitro.

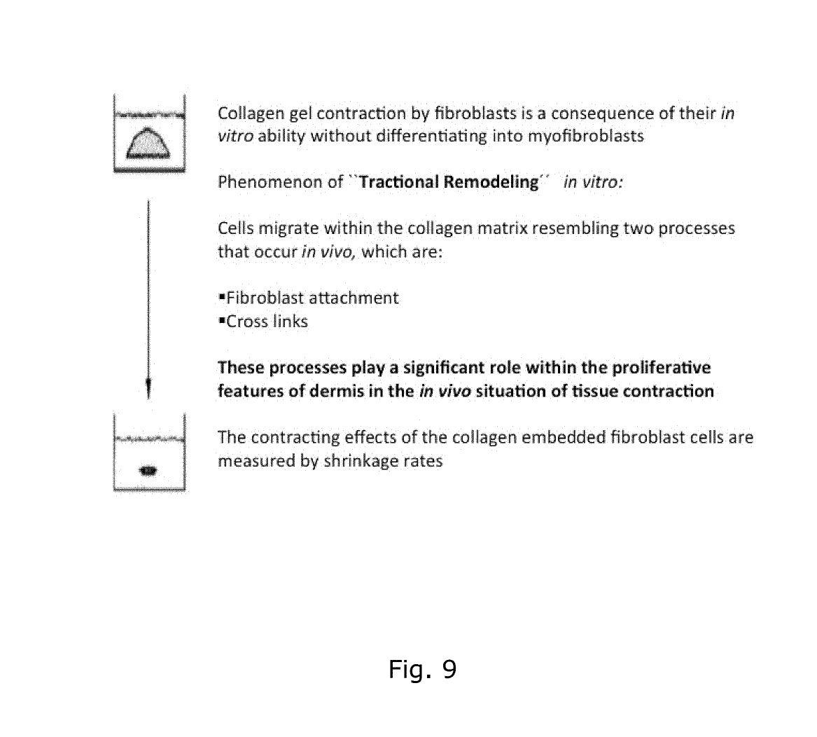

FIG. 9 illustrates the principle of the free floating collagen lattice assay.

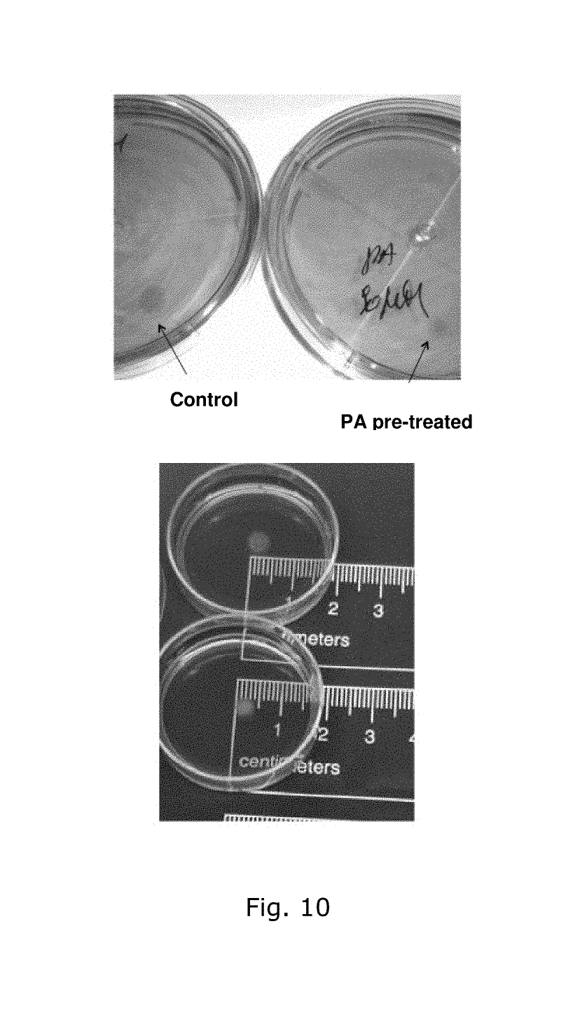

FIG. 10 shows on example of a free floating collagen lattice assay and the effect or pre-treatment of the embedded fibroblast cells with PA. Top: Control and PA pre-treated. Bottom: Measurement of the collageneous lattices upon contraction.

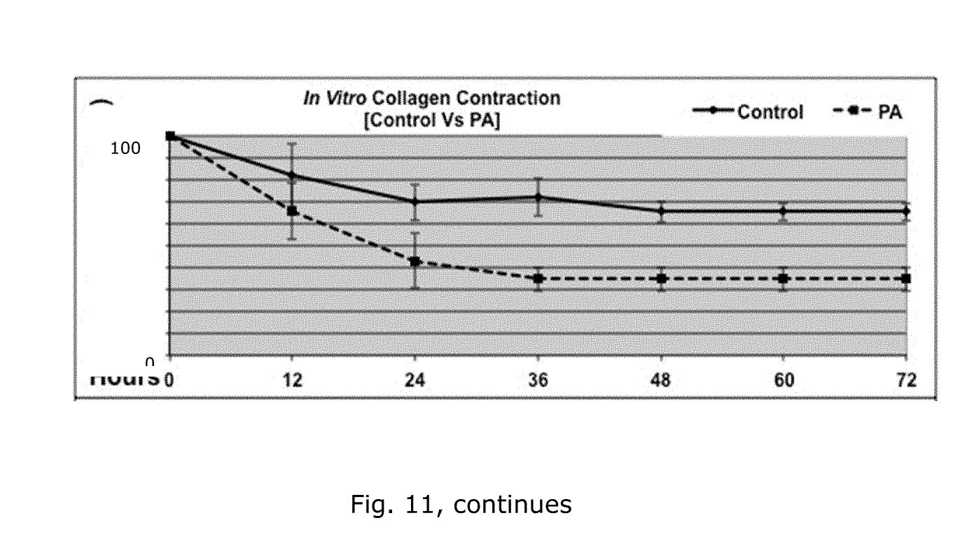

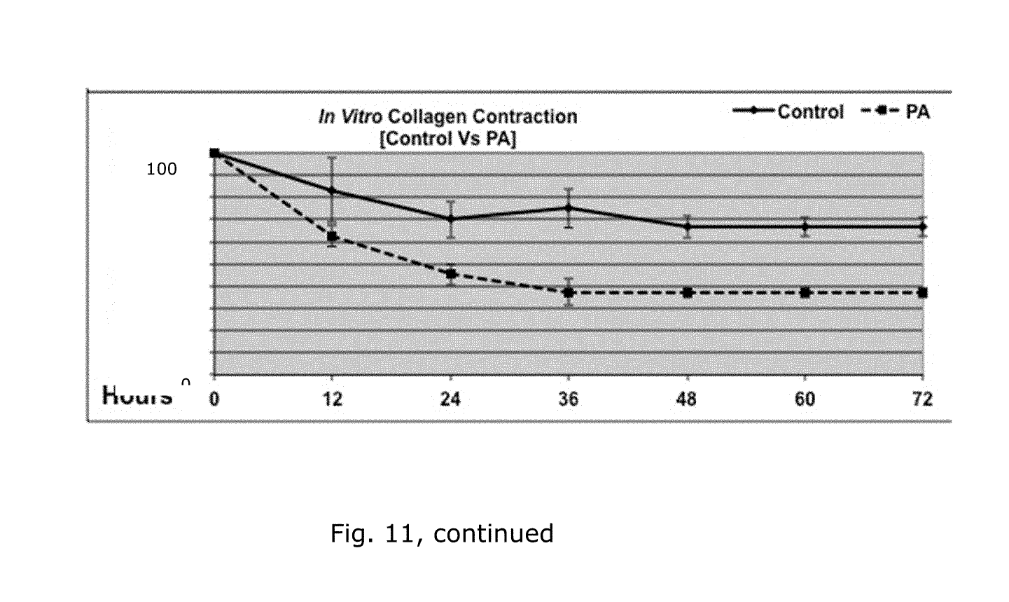

FIG. 11 shows the quantitative results of several free floating collagen lattice assays. X-axis: Hours. Y-axis: Surface area in %.

Graphs show the extent of collagen lattice contraction by PA from two independent experiments. PA improves collagen contraction by 85%.

The present invention will now be described in more detail in the following.

### DETAILED DESCRIPTION OF THE INVENTION

### Repair of Tissue Damage

In a first aspect the invention relates to a compound of the formula (I) or a composition comprising a compound of the formula (I):

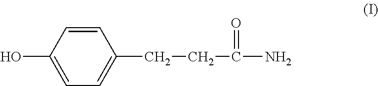 [SMILES: NC(=O)CCc1ccc(O)cc1]

for use as a tissue repair agent and/or, a tissue restitution agent e.g. muscle restitution of athletics. As shown in example 2, the migration/motility assays, clearly shows improved migration after addition of PA, clearly indicating improved tissue repair.

Thus, cells treated with PA obviously obtain much improved abilities to move in to cover up damaged tissue. This having an effect not only on open wounds and soars but most likely also on weaker tissue damage, such as torned and abused muscle fibers

In the present context, 3-(4-hydroxyphenyl)propanoic acid amide corresponds to a compound of the formula (I)

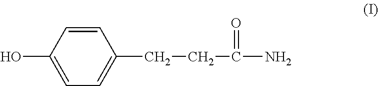 [SMILES: NC(=O)CCc1ccc(O)cc1]

The compound of formula (I), may in here also be denoted as PA. The compound of the formula (I) is also known as (4-hydroxyphenyl)propanoic acid amid, 3-(P-hydroxyphenyl)propionamide or simply phloretamide.

Phrased in another way, the invention thus, also relates to the use of compound of the formula (I) or a composition comprising a compound of the formula (I):

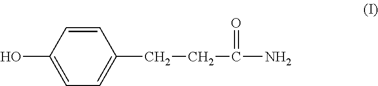 [SMILES: NC(=O)CCc1ccc(O)cc1]

in the manufacture of a medicament for use as a tissue repair agent, a tissue reconstitution agent e.g. muscle restitution of athletics.

The compound or composition may be provided by different routes to a person. Thus, in an embodiment said compound or composition is topically, systemically and/or orally administrated.

In yet an embodiment, said compound or composition is topically applied to a region of the skin, which considered at need of tissue repair.

In a further embodiment, said tissue considered at need of tissue repair is a wound, an operational scar, damaged tissue, such as internal or external, inflammatory scars, an ulcer, a sunburn, a burn wound, bedsores, and/or diabetes wounds.

The provision of the compound and or composition to the skin may be aided in different ways. Thus, in a further embodiment, said topical application is aided by a bandage or patch.

In another embodiment, said compound or composition is provided to a mammal such as a human.

The composition may also be provided in different forms. Thus, in an embodiment, said composition is in the form of a lotion, an emulsion, a crème, an ointment, a stick, a solution in organic solvents, a pack, tonic or a gel.

### Skin Brightening Agent

The compound or composition may also find use as a skin brightening/lightening agent. Thus, in another aspect, the invention relates to the use se of a compound of the formula (I) or a composition comprising a compound of the formula (I):

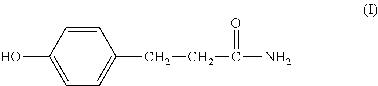 [SMILES: NC(=O)CCc1ccc(O)cc1]

as a skin brightening agent.

In the present context “skin brightening agent” or “skin lightening agent” or “skin whitening agent” refer to a compound or composition able to brighten the skin. PA is able to inhibit tyrosinase, which in mammals (such as humans) is an essential part of the melanin synthesis. Thus, the effect may be due to lowering the level of melanin present in the skin.

In yet an aspect, the invention relates to a compound of the formula (I) or a composition comprising a compound of the formula (I):

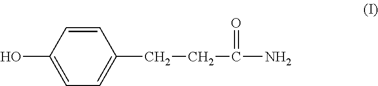 [SMILES: NC(=O)CCc1ccc(O)cc1]

for use as a skin brightening agent.

In yet a further aspect, the invention relates to the use of compound of the formula (I) or a composition comprising a compound of the formula (I):

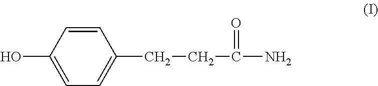 [SMILES: NC(=O)CCc1ccc(O)cc1]

in the manufacture of a medicament for use as a skin brightening agent.

The composition may also be provided in different forms. Thus, in an embodiment, said composition is in the form of a cosmetic, a lotion, an emulsion, a crème, an ointment, a stick, a solution in organic solvents, a pack, a tonic or a gel.

In a more specific embodiment, said compound of the formula (I) is present in the composition at a concentration in the range from 0.01 to 50 wt %, such as 1 to 20 wt %, or such as 5 to 10 wt %.

In another embodiment, the composition further comprises one or more ingredients selected from the group consisting of oil substances, humectants, thickeners, preservatives, emulsifiers, medical ingredients, perfumes, emulsion stabilizers, allantoin, vitamin E acetate, glycyrrhizin, salicylic acid, urea, coix seed, and UV absorbers.

The compound or composition may be provided to a subject by different routes. Thus, in an embodiment said compound or composition is topically applied.

In yet an embodiment, said compound or composition is topically applied to a region of the skin, considered in need of skin brightening/lightening, e.g. a region, which is darker than surrounding regions of the skin.

In a further embodiment, said compound is applied to freckles, chloasma, moth patch, scars and/or pigmentary deposits after sunburn.

In yet another embodiment said topical application is aided by a bandage or patch.

The compound or composition is preferably provided to humans. Thus, in an embodiment, said compound or composition is applied to a mammal such as a human.

When the compound or composition is for oral administration, it may come in different forms. Thus, in an embodiment, said orally administration is in the form of a food, a food ingredient, a neutraceutical, a neutracosmetic, a pill or capsule.

### Tyrosinase Inhibitor

As shown in example 3, the compound or composition according to the invention, also function as a tyrosinase inhibitor. Thus, in an aspect, the invention relates to the use of a compound of the formula (I) or a composition comprising a compound of the formula (I):

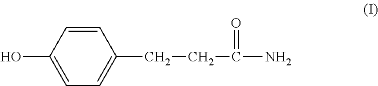 [SMILES: NC(=O)CCc1ccc(O)cc1]

as a tyrosinase inhibitor. Tyrosinase inhibition may also be relevant in non-medical and non-cosmetical fields. For example, tyrosinase inhibition may be relevant to avoid e.g. browning of food such as vegetables and fruit.

### Cell Migration/Motility

As shown in e.g. example 2, the compound or composition according to the invention improves cell migration.

Thus, in an aspect, the invention relates to a compound of the formula (I) or a composition comprising a compound of the formula (I):

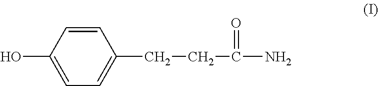 [SMILES: NC(=O)CCc1ccc(O)cc1]

for use as an agent to improve cell motility and/or surface cell migration. In the present context “surface cell migration” relates to the migration of cell on a surface such as a matrix, e.g. cell matrix.

In a further similar aspect, the invention relates to the use of a compound of the formula (I) or a composition comprising a compound of the formula (I):

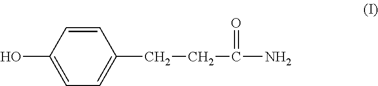 [SMILES: NC(=O)CCc1ccc(O)cc1]

for use as an agent to improve cell motility and/or surface cell migration. Without being bound by theory, it is believed that the improved cell migration/motility is due to improved adhesion to the surface. This is also considered an important feature to avoid e.g. cancer cells escaping from their primary location into the circulatory system and thereby result in metastasis of a cancer. Thus, in an aspect the compound or composition may also be used for improvement of (in vivo and in vitro) cell adhesion.

Such use may also be ex vivo use or in vitro use. Thus, in an embodiment, said use is in vitro (or ex vivo). In a more specific embodiment, said in vitro use (or ex vivo) is for growth of tissue, such as skin or organs.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Methods

### Distinct Mechanism of Fibroblasts in Dermis Equivalents

### Free Floating Collagen Lattice

Aim: to study contraction of ECM molecules by fibroblasts

### Protocol:

Collagen type I from bovine skin can be purchased from IBFB Pharma GmbH, Leipzig, Germany and is prepared according to the distributed protocols.

Cells are trypsinized, suspended in complete growth medium and carefully mixed in the collagen solution (collagen in 0.9×DMEM, 10% FCS, 0.2 mM NaOH, prepared at room temperature). Final cell concentrations might range from 0.8×10⁴ to 3×10⁵ cells/ml lattice. Final concentration of collagen might range from 0.3-0.6 mg/ml lattice.

After mixing, the solution is placed onto bacteriological Petri dishes and immediately put back into the incubator.

Half an hour after polymerization in the incubator, the lattices are carefully detached from the borders of the dishes using a pipette tip. It is important that the Free Floating Collagen Lattices “swim” freely in the medium.

Measurement of the diameter is performed at different time points depending on cell type and seeded cell number. Measurement can be done using a scale paper. From the diameter the area of the collagen lattice can be calculated and their size can be compared at different time points.

Production of collagen lattices:
Estimated time for 20 lattices=1 h conduction plus 1 h until medium addition.
Per assay, 20 empty 35 mm cell culture dishes were pre-warmed to 37° C. The type of plastic dish used may considerably influence attachment strength of the gels.
Cells were trypsinized, and resuspended at ˜10.0×10⁵ cells in 2.5 ml of media. Cell starting number has to be adjusted to the cell type and time of growth.
Cell suspension was stored on ice during preparation of collagen solution.
Collagen solution was prepared on ice from cold and sterile components:

| Components | Amount | Concentration | Manufacturer |
|---|---|---|---|
| rat tail | 1250 μl | 2.3 mg/ml | First Link (UK) Ltd, |
| collagen I | | | RTC1833 |
| (D)MEM (I0X) | 150 μl | 10X | Life Tech Cat. |
| | | | No. 2/430-012 |
| NaOH (sterile) | ca. 100 | 1 N | |

Total: 1500 μl
Collagen I comes dissolved in 0.6% acetic acid from First Link. Any other rat tail collagen (e.g. Sigma) may be used, but protocol has to be adjusted for acetic acid and collagen stock concentration.
NaOH was added drop wise (ca. 8-9 drops from a 100 μl) yellow pipette tip to neutralize the acetic acid. At pH 7.4 phenol in medium turns from yellow to pink and collagen starts to polymerize, work rapidly to prevent formation of collagen aggregates or gelling.
The prepared collagen solution (1.5 ml) was gently mixed with the cell solution (2.5 ml) at RT, resulting in a final cell concentration of 2.5×10⁵ cells/ml and final collagen concentration of 0.72 mg/ml. Again, you may vary the collagen concentration from 0.5 mg/ml (compliant) to ˜2 mg/ml (stiff) by adapting the protocol.
200 μl of collagen/cell mixture were slowly pipetted as a drop onto the center of one prewarmed 35 mm cell culture dish and polymerized at 37° C. for 60 min. A void shaking of the dish to prevent outflow of the droplet and to keep nice circular drop morphology.
After 60-180 min, 2 ml of media were added and the lattices were incubated for up to 5 days without medium change.

### Boyden Chamber:

### To Study Chemotaxis-Directed Mobility of Fibroblast Cells Toward a Chemical Bait

Neuro Probe AP48

48-well Microchemotaxis Chamber Protocol

NOTE: The following directions assume that you are working with polycarbonate filters.

### Preparing the Chamber

1. Adjust a variable-volume micropipette with a 1 mm tip so that the ejected liquid fills a bottom well. The well will hold 25 to 26 μL. A slight positive meniscus should form when the well is filled; this prevents air bubbles from being trapped when the filter is applied.
2. Orient the bottom plate on a flat surface so that the NP trademark is at the upper left. Warm chemo-attractants or control reagents to about 37° C. and de-gas them by vortex or vacuum. Fill the bottom wells, completing the 48 wells in no more than 5 minutes, to prevent excessive evaporation.
3. Cut 1 mm off the corner of a filter membrane and orient it with the cut corner at the upper left. Lift the filter by the ends with two forceps, hold it evenly over the filled wells, and lower it onto them, allowing the middle portion of the filter to make contact first. The filter position can be adjusted at this point if necessary, but note that too much movement will cause contamination between wells.
4. Apply the silicone gasket with its cut corner at the upper left, then the top plate, aligning its NP trademark with the trademark on the bottom plate. Push the top plate down firmly and do not let go; this helps prevent air bubbles from being drawn in and trapped in the bottom wells. With your free hand, apply and tighten the thumb nuts until finger tight. Do not use pliers or other tools to tighten them.

### Preparing and Adding Responding Cells

1. In the upper wells the concentration of cells in the suspension should be adjusted so that 50 μL contains the desired number of cells for one well. For example, since the exposed filter area for each well is 8 mm², a suspension of 8,000 cells in 50 μL will yield 1,000 cells/mm². 50,000 cells in 50 μL will yield approximately 6,000 cells/mm².
2. Pipette cell suspension into each upper well, adjusting the volume so that the filled wells have a slight positive meniscus. Hold the pipette at a steep angle so that the end of the pipette tip rests against the wall of the chamber just above the filter, and the side of the tip rests against the top rim of the well. Eject the fluid with a rapid motion to dislodge air in the bottom of the well.
3. Check for trapped bubbles in the upper wells. One easy way to do this is to look at the reflections of overhead lights in the meniscuses: a well with an abnormally large positive meniscus usually has a trapped air bubble. To remove any bubbles, suck the well completely dry with a suction line and disposable pipette tip, then refill it.
4. For most chemotaxis assays the filled chamber is incubated at 37° C. in humidified air with 5% CO₂. Incubation times vary considerably depending on cell types and chemotactic factors. One good way to determine the optimum incubation time is to use 6 to 12 blind-well chambers (e.g. stock # BW100) set up as negative controls and placed simultaneously in the incubator. Remove one blind-well chamber after a set period (e.g. 30 minutes), and remove the rest sequentially, one every 5 minutes. Stain the filters and examine them to see how long unstimulated cells have taken to migrate through the filter, or, if you are using cellulose nitrate filters, to a specified optimum depth.

### Staining Polycarbonate Filters

1. Aspirate fluid from the top wells or empty them by shaking the chamber over a sink or container.
2. Remove the thumbnuts while holding down the top plate, and invert the entire chamber onto a paper towel. Grasp the four corners of the top plate (now on the bottom) and push down evenly so that it stays level as it drops to the table. If the gasket should hang up on the post hardware, carefully push it down evenly onto the plate. Take care not to touch the filter, which should be stuck to the gasket. Immerse the remaining plate (with stud hardware in place) in cool distilled water.
3. The migrated cells are now facing up on the filter; this side of the filter is henceforth referred to as the cell side. Lift up one end of the filter with forceps and catch 1 mm of the edge in the large filter clamp. Lift the filter and quickly attach the small filter clamp to the edge of the free end. Keeping the cell side up, wet the underside (non-migrated cell side) of the filter in a dish containing PBS. Do not let the PBS wash over the cell side of the filter.
4. Holding the filter by the large clamp, with the small clamp attached to the other end and hanging free, wipe the cells off the non-migrated cell side of the filter by drawing the filter up over the wiper blade. The blade should first contact the filter just below the jaws of the wide clamp. Use only gentle pressure against the blade, and maintain an angle of about 30° from the vertical for the portion of the filter above the wiper. It is important to complete the wiping carefully and quickly so that the cells will not dry on the filter; drying takes place in 10 to 20 seconds, and will prevent complete removal of the non-migrated cells.
5. Clean the wiper with a Q-Tip, again wet the underside of the filter in PBS, and repeat Step 3. Clean the wiper again, then wet the filter a third time in PBS, and repeat Step 3.
6. For granulocytes and monocytes, carefully immerse the filter in methanol, then place the filter cell-side up on a disposable lint-free towel for air-drying. Rinse all chamber components in cool distilled water. For other kinds of cells, consult the literature for staining techniques.
7. When the filter is dry, clamp the edge of one end with a large filter clamp, weight the other end with a small filter clamp, and stain in Diff-Quik® or equivalent dye, according to the manufacturer's instructions. To avoid contaminating the chamber components with stain, it is convenient to have two sets of filter clamps, one for removing the filter from the gasket, and one for staining.
8. Place the wet filter cell-side up on a 50×75 mm microscope slide to dry.

When the filter is dry, center it on the slide and place a drop of immersion oil on it. Rub the oil over the filter with a smooth, blunt instrument to remove all bubbles and wrinkles. The filter is now ready for counting.

### Example 2—Uni and Bi Directional Cell Migration

### Aim: To Determine Effects of 3-(4-Hydroxyphenyf)Propanoic Acid Amide Treatments on Fibroblasts Migration.

### Introduction

Delicate cell monolayers at confluency are mechanically disrupted leaving an area devoid of cells. This procedure is accomplished by a “scrape” made on the cells and later advancement of the adjacent cells into the open area by cell mechanical migration, which is monitored by microscopy. Depending on the cell type, the covering process can take from many hours to less than a day, which is entirely dependent on the type of cells and extent of the scrape. The rate or the extent of migration of the cells (often termed as repopulation rate) can be calculated by a series of photomicrographs.

### Protocol

Plate fibroblasts at a density 1×10⁴ in 35 mm tissue culture plates and incubate for three days using novel test compounds with a control at 37° C., 5% CO₂.
At confluency, displace a group of cells within the monolayer by making one streak using a sterile pipette tip.
Photograph under phase contrast microscopy sequentially at an interval of 19 hrs post scraping.
Using an ocular micrometer measure the scrape size at each time point.
Evaluate statistical significance by Student's t test.
Observe significant differences in scrape repairin PA pre-treatment on fibroblasts.

### Results

FIG. 1 shows a general overview of which in vivo processes are reflected by the in vitro assays employed in the presented experiments.

The results of the bi-directional scratch assays presented in FIGS. 2 and 3, clearly show improved cell migration when PA is added.

The results of the uni-directional scratch assays presented in FIGS. 4 and 5, clearly show improved cell migration when PA is added.

The results of the chemotactic migration assays (Boyden Chamber assay) presented in FIGS. 7 and 8, clearly show improved cell migration when PA is added. The Boyden Chamber assay is schematically illustrated in FIG. 6.

The results of the free-floating collagen lattice assays presented in FIGS. 10 and 11, clearly show improved cell migration/motility when PA is added. The free-floating collagen lattice assay is schematically illustrated in FIG. 9.

### Conclusion

The results obtained by employing this collection of cellular migration assays clearly demonstrate a significant improvement of cell motility in PA-treated cells, indicating that PA potentially strongly improve human cell's ability to repair imposed tissue damage.

### Example 3—Skin De-Pigmentation Assay

### To Study the Effects of 3-(4-Hydroxyphenyf)Propanoic Acid Amide on Melanin Production in Human Cells

### Introduction

Melanin pigments, the determinants of skin color are produced by Melanocytes located on the basal layer separating the dermis and epidermis in the skin of the human body. The enzyme Tyrosinase is responsible for catalyzing various steps in the melanin pigment biosynthetic pathway responsible for skin cell pigmentation. Accordingly, Tyrosinase inhibition promotes skin de-pigmentation (skin-lightning).

### Aim of the Study

The present study is aimed at investigating the skin lightning effects using an animal model system in vitro

### Assay Standardization

The assay was standardized using B10F16 mouse melanocytes with cell numbers at a density ranging from 5×10⁴ to 1×10⁵ cells per ml. It was found that 10⁵ cells were sufficient to get enough cell pellet and the cells were seeded in Petri dishes in equal numbers. The following day the cells were pre-treated with 80 μM of PA and 10 μM+control for 2 h, 24 h, 72 h and 96 h.

Cells were then collected, counted for cell number and equal numbers were pelleted. The cell pellet was visually observed for colour intensity and quantified. The same cell pellet was then used for protein extraction and immunoblot analysis.

### Materials and Methods

Test solution preparation: Stock solutions of the test compound are prepared by dissolving in DMSO. The stock solutions were filter sterilized, stored in fridge at 4° C., and were used for experiments by dilution it in the cell culture medium as required.

### Cell Lines:

Mouse melanocytes (B10F16 cell line) were maintained in Dulbecco's modified Eagle's medium (DMEM; Invitrogen) supplemented with 10% foetal bovine serum (v/v; Invitrogen), 2 mM glutamine, and 1% non-essential amino-acids.

### Culture Conditions:

Cells were sub-cultured at 37° C., 5% CO2 and 95% humidity, were fed approximately 16 h prior to each assay and cell number was determined in duplicates using a Coulter Z2 counter (Beckman Coulter). Mouse melanocytes were sub-cultured when they reached confluence at a split ratio 1:4.

### Bradford Assay for Protein Estimation:

Stock solution of concentration 1 μg per μl was made from BSA.

### Assay Reagent—

The assay reagent is prepared by diluting 1 volume of the dye stock with 4 volumes of distilled H₂O. The solution is stored in a dark bottle at 4° C.

### Protein Standards—

Protein standards were prepared in the same buffer as the samples to be assayed. A convenient standard curve can be made using bovine serum albumin (BSA) with concentrations of 0, 2, 4, 6, 8, 10, 15 and 20 μg/mL for the micro assay.

### Standard Protein Assay Procedure:

Eight standard solutions (1 mL each) containing 0, 2, 4, 6, 8, 10, 15 and 20 μg/mL BSA were studied. The spectrophotometer was set to a wavelength of 595 nm. 4 mL plastic cuvette filled with distilled water to blank the spectrophotometer was used over this wavelength. The plastic cuvette was emptied into a test tube and shaken out any remaining liquid. 4 μL of protein standard solution and 1 mL of assay reagent were added, starting with the lowest protein concentration and the samples to be assayed. The absorbance spectrum of the sample from 400 to 700 nm was recorded, and the absorbance at 595 nm was noted. Steps were repeated above for each of the protein standards and for the samples to be assayed. The spectra of the standards and samples were examined. To determine the protein concentration of a sample from it absorbance, use the standard curve to find the concentration of standard that would have the same absorbance as the sample.

### Results:

| −C | Control no compound |
|---|---|
| +C | Positive Control (Hydroquinone at 10 μM) |
| PA | Compound at 80 μM |
| | After 96 hrs |
| − C | Standard dark |
| +C | 78% lighter (78% relative to −control) |
| PA | 39% lighter (relative to +control) |
| PA | 50% lighter (relative to − control) |

Hydroquinone is a well-known tyrosinase inhibitor albeit with known health risks.

### CONCLUSION

The compound PA showed distinct color change from dark to pale indicating significant effect on inhibition of melanin production.

1. A compound of the formula (I) or a composition comprising a compound of the formula (I): 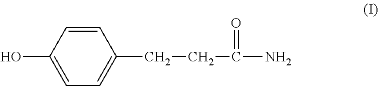 [SMILES: NC(=O)CCc1ccc(O)cc1]
for use as a tissue repair agent.

2. The compound according to claim 1, wherein said composition is topically, systemically, and/or orally administrated.

3. The compound or composition according to claim 1, wherein said compound or composition is topically applied to a region of the skin, which considered at need of tissue repair.

4. The compound according to claim 1, wherein said tissue considered at need of tissue repair is a wound, an operational scar, damaged tissue, such as internal or external, inflammatory scars, an ulcer, a sunburn, a burn wound, bedsores, and/or diabetes wounds.

5. The compound or composition according to claim 1, wherein said topical application is aided by a bandage or patch.

6. The compound according to claim 1, wherein said composition is in the form of a lotion, an emulsion, a crème, an ointment, a stick, a solution in organic solvents, a pack, tonic or a gel.

7-16. (canceled)

17. Use of a compound of the formula (I) or a composition comprising a compound of the formula (I): 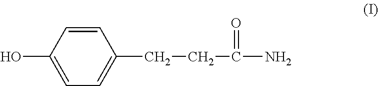 [SMILES: NC(=O)CCc1ccc(O)cc1]
for use as an agent to improve cell motility and/or surface cell migration.

18-19. (canceled)
